(19) United States
(12) Patent Application Publication
(10) Pub. No.: US 2025/0186706 A1
(43) Pub. Date: Jun. 12, 2025

(54) Treatment Apparatus and Method to Enhance Performance of Medical Needles and Sharp Instruments

(71) Applicant: Retractable Technologies, Inc., Little Elm, TX (US)

(72) Inventors: Thomas J. Shaw, Frisco, TX (US); Jordan Duesman, Pilot Point, TX (US)

(21) Appl. No.: 18/971,844

(22) Filed: Dec. 6, 2024

Related U.S. Application Data
(60) Provisional application No. 63/607,775, filed on Dec. 8, 2023.

(51) Int. Cl. A61M 5/32

(52) U.S. Cl. CPC A61M 5/3221; A61M 5/3286; A61M 5/3287; A61M 2205/19

(57) ABSTRACT

Systems and methods are disclosed that include providing a syringe with an enhanced needle, at least part of which is subjected to a treatment that enhances the performance of the needle. The treatment is administering at least a portion of the needle by projecting a heated, ionized airflow over the at least a portion of the needle at a temperature of at least 225° F. to not greater than 325° F. for not greater than 1 second. The treatment reduces a post-draw needle penetration force of the needle as compared to a traditional untreated needle, enhances lubrication adherence to the needle, and reduces residual metallic particle contamination resulting from the manufacturing process when compared to a traditional needle that has not been subjected to a surface treatment, which result in a significant reduction in the pain patient may experience during an injection.

### CROSS-REFERENCE TO RELATED APPLICATIONS

This application claims priority under 35 U.S.C. § 119(e) to U.S. Provisional Patent Application No. 63/607,775 filed on Dec. 8, 2023 by Thomas J. Shaw et. al. and entitled “Treatment Apparatus and Method to Enhance Performance of Medical Needles and Sharp Instruments,” the disclosure of which is hereby incorporated by reference in its entirety for all purposes.

### BACKGROUND OF THE INVENTION

Medical syringes having a needle have been used for administering injections or infusions of drugs or medicines. The process of administering an injection typically involves inserting the needle of a syringe through an elastomeric seal of a vial to draw up the drug, medicine, or other fluid into the syringe. After removing the syringe from the vial, the drug, medicine, or other fluid from the syringe via injection into a patient or subject. Many people experience discomfort and/or pain when receiving injections.

Such discomfort or pain can often be attributed to, or made worse by, a bent, compromised, or otherwise damaged needle point or tip that may result from the insertion of the needle through the elastomeric seal of the vial required for drug, medicine, or other fluid draw. In an effort to ameliorate or lessen the discomfort attributable to bent, compromised, or otherwise damages needle tips, the drawing needle is frequently replaced with a new needle (one that has not been previously inserted through the elastomeric seal or otherwise used), prior to the injection. This practice is wasteful of a medical professional's time in having the change out the drawing needle for a new needle and wasteful of needle resources. Waste of needle resources can be particularly problematic when medical needles are in high demand, for example when there was a need for a large quantity of needles to provide vaccinations for COVID-19.

Such discomfort or pain can often be attributed to contamination on the needle surface, such as residual metal particulates from the needle manufacturing process, or from an uneven or rough surface resulting from the needle manufacturing process. In order to reduce roughness and smooth a needle surface, U.S. Pat. No. 9,265,896 discloses a specialized plasma treatment process. The rough needle is placed in a vacuum chamber with an electrode to produce a plasma created by introducing argon, or a mixture of argon and air, into the vacuum chamber while supplying 50 to 1000 V to the electrode to create an arc discharge with temperatures over 450° F. The needle is processed with the plasma in the vacuum chamber for at least 1 hour and up to 50 hours. While the example results in the '896 patent indicate positive results in achieving a reduction of penetration value of the plasma treated needles, this process is not commercially viable because the need for a vacuum chamber and the amount of time required for the plasma treatment would make the needles far too expensive to produce. A needle that normally costs around $0.00466 to produce in around 0.3 seconds would substantially increase the amount of a final product if the process could not keep pace with high-speed production (greater than 2 syringes per second). This would explain why the process described in the '896 patent is not being utilized by major syringe manufacturers. Additionally, by using specialized gases (such as argon) and applying high voltages, there are increased hazards for workers in the plasma treatment process area.

Other sharp instruments, such as scalpels and scissors, have also been used for medical procedures requiring incisions or cutting through bodily tissue. These types of sharp instruments can experience similar issues with particulate contamination and bending or dulling from a first incision or cut.

Thus, the medical industry continues to demand advances in needle and other sharp instrument technology to reduce and/or eliminate damage to needle tips and sharp edges during use and overall increase the structural integrity of needle tips and sharp edges.

### SUMMARY

According to one preferred embodiment of the proposed application, a method of treating a medical needle or a medical sharp instrument comprises exposing a least a portion of a surface of the medical needle or the medical sharp instrument to an airflow for a period of time of less than 1 second. Most preferably, an airflow comprises one or both of the following characteristics: (1) it is pressurized to a pressure over ambient pressure and/or (2) it is heated to above ambient temperature. In another preferred embodiment, an airflow further comprises a characteristic of being ionized.

In another preferred embodiment, an airflow is made from ambient air treated to have one or more of the above characteristics. References herein to “ambient air” refer to the composition of air as untreated, breathable air (e.g., having around 78% nitrogen, around 21% oxygen, and small amounts of argon, carbon dioxide and other gasses), without reference to ambient temperature or ambient pressure. No specialized gases are needed to generate an airflow and no other treatments, such as purification (to increase a concentration of one type of gas) or dehumidifying are required. By not requiring the use of specialized gases, such as argon, methods herein are safer for workers around an area where a treatment will be carried out, do not require delivery and storage of potentially dangerous and expensive gas canisters, and to not require specialized equipment to purify ambient air to produce a higher concentration of one component (e.g., oxygen). In still other preferred embodiments, ambient air may be filtered to remove particulates or dust and/or may have its humidity level adjusted to generate an airflow.

In one preferred embodiment, an airflow is heated to above ambient temperature using a heating element. In one preferred embodiment, a heating element generates heat through resistance and an airflow is heated by passing ambient air over and/or around the heating element. In another preferred embodiment, a heating element comprises at least one electrode to which a voltage is applied and an airflow is heated and ionized by passing ambient air over and/or around the at least one electrode. In still another preferred embodiment, no electrical current ionization is applied to generate the airflow and only resistance heating is applied to the ambient air. In still another preferred embodiment, a heating element comprises a flame from burning a gas, such as a gas comprising methane and/or propane.

In still other preferred embodiments, a treatment system or a treatment apparatus generates an airflow. A treatment apparatus may comprise a housing and may comprise a nozzle to aid in directing an airflow to a surface being treated. In some embodiments, a housing of a treatment apparatus is connectable to an external air compressor in a treatment system or a treatment apparatus may comprise an internal compressor. In other embodiments, a treatment apparatus may comprise an electrode and/or a heating element within a housing. A treatment apparatus preferably does not require a sealed housing or a vacuum chamber to generate an airflow for treatment.

In some embodiments, a treatment system or apparatus is stationary relative to a surface being treated and an airflow is projected or blown onto the surface as the surface passes by the treatment apparatus, preferably a nozzle on a treatment apparatus. In other embodiments, a surface to be treated in stationary relative to a treatment system or treatment apparatus and an airflow is projected or blown onto the surface as the treatment system or treatment apparatus, preferably a nozzle on a treatment apparatus, passes over the surface. In still other embodiments, a surface to be treated and treatment apparatus (or a treatment system) both move relative to each other to project or blow an airflow onto the surface.

In some embodiments, an airflow is heated (with or without electrical charge ionization) to a temperature within a range of at least 225° F. to not greater than 325° F. In still other embodiments, an airflow is pressurized to a pressure of around 70 to 90 psi. In other embodiments, an airflow is applied to a surface being treated using a treatment apparatus and/or a treatment method herein for at least 0.05 second to not longer than 0.90 seconds.

A preferred embodiment of a treated needle herein has a reduced penetration force upon a second insertion (inserted two times, generally first through an elastomeric seal on a medicine vial to draw medicine and second through a patient's skin to inject the medicine) as compared to a second insertion needle penetration force a conventional needle without treatment. In other preferred embodiments, a treated needle comprises less contamination and particulate matter as compared to a conventional needle without treatment. In other preferred embodiments, a treated needle provides a surface more conducive to the adhesion of lubrication than a convention needle without treatment. In still other preferred embodiments, a needle tip of a treated needle is less likely to be bent or damaged after a first insertion, making it usable for a second insertion. A treated needle is preferably one produced with a treatment apparatus and/or treatment method according to preferred embodiments herein. Other than subjecting at least a portion of the needle to an airflow having the characteristics described herein and for a treatment period of 1 second or less, no other special processing or materials are required to manufacture a treated needle. Any commercially available medical needle may be treated as described herein to be a treated needle.

In some embodiments, a portion of a needle that is treated with an airflow comprises a needle tip. In other embodiments, a portion of a needle that is treated with an airflow comprises a needle tip and a bevel. In still other embodiments, a portion of a needle that is treated with an airflow comprises a needle tip, a bevel, and a forward portion of a shaft. In other embodiments, an entirety of a needle is treated with an airflow. A needle may be treated with an airflow before or after being inserted into a needle hub, a syringe, or other needle holding device.

Producing a treated needle according to embodiments herein, particularly preferred embodiment using a treatment apparatus and/or treatment method herein, results in performance enhancing attributes compared to a conventional needle that is not treated. These attributes include a reduced second insertion penetration force, less contamination and particulate matter from the needle manufacturing process, greater adherence of lubricant applied to the needle, and/or a needle tip that is less likely to be bent or damaged after a first insertion. A treated needle may be used for at least two insertions (one draw and one injection), eliminating the waste of time and needle resources attributable to replacing a draw needle with a new needle before an injection. A treated needle may also be produced in-line with a needle manufacturing process, allowing needles to be treated at high speeds of a production line without any significant increase in production time.

### BRIEF DESCRIPTION OF THE DRAWINGS

For a more complete understanding of the present disclosure and so that the manner in which the features and advantages of the embodiments can be understood in more detail, reference is now made to the following brief description, taken in connection with the accompanying drawings and detailed description.

FIG. 1 shows an oblique view of a syringe according to an embodiment of the disclosure.

FIG. 2 shows an orthogonal side view of a needle according to an embodiment of the disclosure.

FIG. 3 shows a cross-sectional side view of a needle according to an embodiment of the disclosure.

FIG. 4 shows a needle treatment apparatus for treatment of one or more needles according to an embodiment of the disclosure.

FIG. 5 shows a flowchart of a method of enhancing the performance of a needle according to an embodiment of the disclosure.

FIG. 6A shows an image of a needle tip of a comparative sample of a conventional needle without treatment prior to undergoing a first insertion process.

FIG. 6B shows an image of a needle tip of the comparative sample of the conventional needle without treatment after undergoing the first insertion process.

FIG. 7A shows an image of a needle tip of an exemplary embodiment of a needle according to an embodiment of the disclosure prior to undergoing a first insertion process.

FIG. 7B shows an image of a needle tip of exemplary embodiment of the needle according to an embodiment of the disclosure after undergoing the first insertion process.

FIG. 8A shows a graph of peak force versus tensile extension of a plurality of comparative samples of conventional needles without treatment.

FIG. 8B shows a graph of peak force versus tensile extension of a plurality of exemplary embodiments of needles according to an embodiment of the disclosure.

FIG. 9A shows an image of a needle tip of an exemplary embodiment of a needle prior to undergoing a surface treatment according to an embodiment of the disclosure. The image is overlaid with a graph showing the height or profile of contaminants present on the needle tip prior to undergoing the surface treatment.

FIG. 9B shows an image of the needle tip of the exemplary embodiment of the needle after undergoing the surface treatment according to an embodiment of the disclosure. The image is overlaid with a graph showing the height or profile of contaminants present on the needle tip after undergoing the surface treatment.

FIG. 10 shows an energy-dispersive X-ray spectrometry (EDS) image of a needle tip of a comparative sample of a conventional needle without treatment subjected to a scanning electron microscopy/energy-dispersive X-ray spectrometry (SEM-EDS) analysis.

FIG. 11 shows an energy-dispersive X-ray spectrometry (EDS) image of a needle tip of an exemplary embodiment of a treated needle subjected to a scanning electron microscopy/energy-dispersive X-ray spectrometry (SEM-EDS) analysis.

The use of the same reference symbols in different drawings indicates similar or identical items.

### DETAILED DESCRIPTION OF PREFERRED EMBODIMENTS

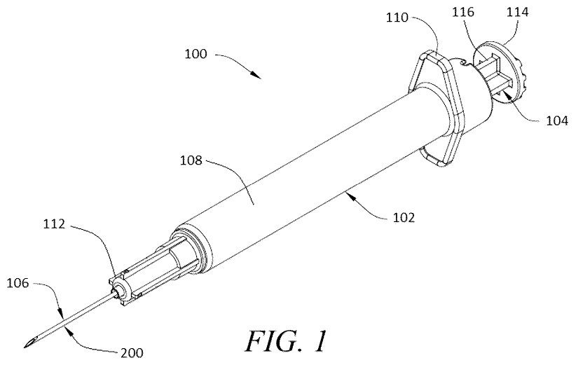

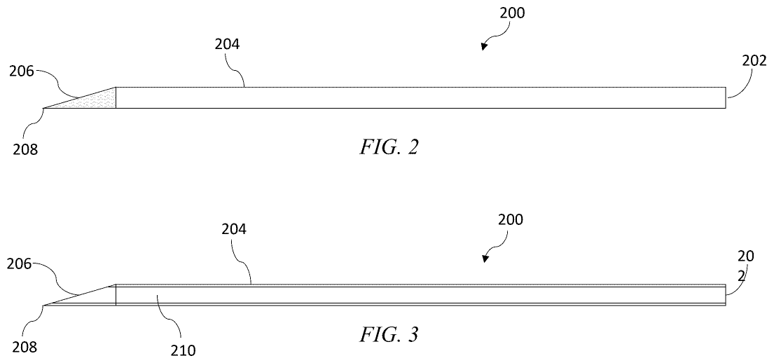

FIG. 1 shows an oblique view of a syringe 100 comprising a treated needle 200 and FIGS. 2 and 3 show an orthogonal side view and a cross-sectional side view of a treated needle 200 according to a preferred embodiment of the disclosure. Treated needle 200 is preferably one that has received a surface treatment on at least a portion of its surface using a needle treatment apparatus 400 and/or a surface treatment method 500 according to embodiments as described herein. In some embodiments, treated needle 200 is made from metal and may generally comprise a base 202 disposed at a rear end, a shaft portion 204, a bevel 206, a needle point or tip 208 disposed at a forward end, and a fluid pathway or lumen 210 extending through an interior of needle 200. In some embodiments, treated needle 200 may be formed from stainless steel or from titanium. In some embodiments, base 202 may comprise a substantially similar diameter as shaft portion 204, such that base 202 and shaft portion 204 comprise a constant diameter. In other embodiments, base 202 may comprise a radially extending chamfer or radiused portion that interfaces with a needle holder of needle assembly 106. Shaft portion 204 may extend between base 202 and bevel 206. Shaft portion 204 may comprise a substantially constant diameter along its length. However, it will be appreciated that the diameter and the length of shaft portion 204 may vary between various needles depending on the intended application.

Bevel 206 may be located at an opposing end of needle 200 from base 202, or on both ends of a two-ended needle such as would be utilized in blood collection tube holder devices. Bevel 206 may comprise a tapered portion, preferably a longitudinally tapered portion disposed on a portion of a circumference of treated needle 200, that tapers to needle tip 208. Bevel 206 may be designed to pierce an elastomeric seal on a vial of medicine or other fluid and the skin of a patient or subject receiving an injection. Bevel 206 may be preferably angled depending on the application and/or the size of the treated needle 200. For example, an intravenous needle for administering a drug, medicine, or other fluid to a human may comprise a bevel 206 with a different angle than a bevel 206 for administering a drug, medicine, or other fluid in a veterinarian application. In some embodiments, bevel 206 may be designed to maximize the integrity or strength of needle tip 208. Lumen 210 may generally extend from base 202 through shaft portion 204 and to bevel 206 to provide a fluid pathway and fluid communication from barrel 102 of syringe 100 through bevel 206. Lumen 210 may comprise a constant diameter (commonly referred to as “gauge” in the art) to promote a laminar fluid flow through treated needle 200 during the administration or injection of a drug, medicine, or other fluid from syringe 100 through treated needle 200.

In addition to treated needle 200, syringe 100 may generally comprise any other components of known prior art syringes for medical purposes. For example, syringe 100 may comprise a barrel 102, a plunger 104, and a needle assembly 106. Barrel 102 may comprise a cylindrical tubular body 108 having one or more projecting finger flanges 110 and a luer tip for receiving needle assembly 106. Plunger 104 may comprise a plunger surface 114 for actuating plunger 104 and an elongated plunger body 116 received within cylindrical tubular body 108 of barrel 102. Plunger 104 may also comprise a plunger seal (not shown) that forms a fluid tight seal between plunger 104 and barrel 102, and more specifically, between a forward end of elongated plunger body 116 of plunger 104 and an interior surface of cylindrical tubular body 108 of barrel 102. Needle assembly 106 may comprise a needle hub 112 configured to receive a needle that is to be treated or to receive a treated needle 200 extending from a forward end of needle hub 112. Needle hub 112 may also be configured to engage with and extend forwardly from a luer tip at a forward end of barrel 102, to provide fluid communication from a fluid chamber insider barrel 102 through to treated needle 200. Needle hub 112 may be configured to retain a needle to be treated or a treated needle 200 in fixed relation to barrel 102 when syringe 100 is fully assembled. Needle hub 112 is preferably configured to be removable from barrel 102, but a treated needle 200, a needle treatment apparatus 400, and/or a surface treatment method 500 herein may also be used with needles for syringes that are configured with retractable needles or needles that are not removable, or not intended to be removable, from the barrel.

A treated needle 200 is a needle, at least a portion of which, has been subjected to surface treatment using a needle treatment apparatus 400 and/or a surface treatment method 500 according to embodiments herein in order to enhance the performance of treated needle 200 over a conventional needle. As used herein references to a “conventional needle” or an “untreated needle” refer a needle that has not been treated with needle treatment apparatus 400 or surface treatment method 500 according to an embodiment herein. For purposes of comparisons to treated needle 200, a conventional needle is preferably one that (1) is made using the using the same materials and processes as treated needle 200 but without the treatment or (2) is a commercially available needle that is similarly sized and configured (such as by use type) as treated needle 200 but without the treatment.

Since the sharp end of treated needle 200 is most prone to contamination and damage, a surface treatment may be applied to bevel 206 and needle tip 208 to produce a treated needle 200. In some embodiments, the surface treatment may impart changes in the properties of a surface of treated needle 200. In some embodiments, the surface treatment may be administered to produce a treated needle 200 prior to lubrication of treated needle 200. Further, in some embodiments, the surface treatment may be administered prior to or after treated needle 200 is installed in needle hub 112 and/or a syringe 100 and/or other medical devices utilizing. It may be evident that a needle is a treated needle 200 herein by a golden-colored tint that may be left on a treated portion of treated needle 200, such as bevel 206 and/or needle tip 208.

It may be further evident that a needle is a treated needle 200 by enhanced performance properties of treated needle 200. In some embodiments, a surface treatment of treated needle 200 may remove residual metallic particulate that remains on the needle from the needle manufacturing process, thereby reducing contamination on treated needle 200 and resulting in a much cleaner treated needle 200. In some embodiments, a pressurized airflow utilized in needle treatment apparatus 400 and/or a surface treatment method 500 according to embodiments herein may blow off residual metallic particulate from the surface of the needle being treated to produce a treated needle 200 with reduced contamination. In some embodiments, a heated and/or ionized airflow utilized in needle treatment apparatus 400 and/or a surface treatment method 500 according to embodiments herein may further reduce contamination by destroying/burning off the contaminants, including biological contaminants In some embodiments, an ionization utilized needle treatment apparatus 400 and/or a surface treatment method 500 according to embodiments herein may cause the residual metallic particulates to release from the surface of the treated needle 200, which may in some embodiments, be aided by a pressurized airflow. In some embodiments, the metallic particulate may be reduced by at least around 1% to around 25%. In other embodiments, metallic particulate may be reduced by at least 1%, at least 2%, at least 3%, at least 4%, at least 5%, at least 10%, at least 15%, at least 20%, at least 25%, or even greater compared to a conventional needle. In some embodiments, a treated needle 200 may also have increased the adherence of a lubricant to the treated needle 200, since the lubricant adheres to clean surfaces better than a contaminated surface. Accordingly, in some embodiments, a treated needle 200 may maintain the lubrication through the process of drawings a drug, medicine, or other fluid from the vial, which may reduce the initial and/or overall penetration force required for treated needle 200 to pierce or penetrate the elastomeric seal of the vial. Further, in some embodiments, the enhanced adherence of the lubrication to a surface of a treated needle 200 may also reduce the initial and/or overall penetration force required for treated needle 200 to pierce or penetrate skin on a patient or subject during administration or injection and may allow the same treated needle 200 to be used for both the draw process and the injection process.

Furthermore, since heat is used in a surface treatment according to a preferred embodiment, treated needle 200 may comprise a greater strength and integrity of needle tip 208 of treated needle 200 as compared to conventional needles that that have not been subjected to a surface treatment. This may greatly reduce and/or altogether eliminate damage (e.g., bending, dulling, etc.) to needle tip 208 during the draw process or during administration or injection of the drug, medicine, or other fluid into a patient or subject. Accordingly, in some embodiments, a strengthened needle tip 208 having a surface treatment according to a preferred embodiment may maintain its integrity and sharpness, thereby reducing the needle penetration force required for the treated needle 200 to pierce or penetrate the skin of a patient or subject after penetration of an elastomeric seal on a medicinal vial as compared to a conventional needle without treatment. In some embodiments, treated needle 200 may reduce the needle penetration force by at least 1%, at least 2%, at least 3%, at least 4%, at least 5%, at least 6%, at least 7%, at least 8%, at least 9%, at least 10%, at least 15%, or even greater over a conventional needle without treatment. This may drastically reduce the amount of discomfort and/or pain patients or subjects often experience with conventional needles during administration or injection. Further, it will be appreciated by those skilled in the art that the contemplated surface treatment may be applied to cannulas, needles, scalpels, or any other sharp medical tools used to penetrate or make incisions in skin of a patient, subject, or other pierceable objects or seals to reduce the required force, thereby reducing the associated discomfort or pain and making penetration or piercing easier.

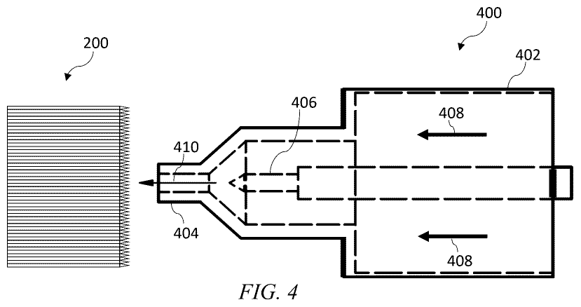

FIG. 4 shows a needle treatment apparatus 400 for treatment of one or more needles to produce treated needles 200 according to an embodiment of the disclosure. Needle treatment apparatus 400 may generally comprise a housing 402 that may be connected to a compressed or pressurized air supply apparatus (not shown) to form a treatment system. A compressed or pressurized air supply apparatus, such as a compressor, is preferably configured to supply a compressed or pressurized airflow 408 (as shown with arrows) through the housing 402. Alternatively, a compressor may be incorporated into housing 402 as part of needle treatment apparatus 400 to draw ambient air into housing 402 and compress it to generate pressurized airflow 408. Preferably, pressurized airflow 408 is at a pressure of around 60 to 110 psia, more preferably of around 70 to 100 psia, and most preferably of around 80 to 90 psia. In some embodiments, pressurized airflow 408 may also be heated above an ambient temperature utilizing an electrode 406 or a heating element.

Housing 402 may comprise and/or taper to a nozzle portion 404 that comprises a reduced diameter as compared to a main body portion or remainder of housing 402. Housing 402 may also comprise one or more electrodes 406 disposed within housing 402, preferably in an upstream location relative to pressurized airflow 408 and nozzle portion 404. In some embodiments, electrode 406 may extend into or close to nozzle portion 404 of housing 402. In operation, pressurized airflow 408 may be passed through the housing 402 and across and/or around electrode 406. An electrical current may be supplied to and/or through the electrode 406. As the pressurized airflow 408 passes across and/or around electrode 406, the electrode 406 may ionize and heat the pressurized airflow 408 to produce a heated, pressurized, and ionized airflow (for ease of reference, referred to herein as ionized airflow 410) that exits the nozzle portion 404. Ionized airflow 410 may be directed to one or more needles to provide a surface treatment to produce treated needles 200. Preferred surface treatment methods of embodiments herein utilize needle treatment apparatus 400; however other apparatus, systems, and devices may be used to provide a surface treatment according to embodiments herein.

In other embodiments, a heating element may be used in place of electrode 406. A heating element may be used to heat pressurized airflow 408 into a heated (and pressurized) airflow that exits nozzle portion 404 like ionized airflow 410, but without any electrical charge ionization of the airflow. In still other embodiments, a heat element may be used in addition to electrode 406 if temperatures higher than those created by electrode 406 are desired.

In some embodiments, needle treatment apparatus 400 may be movable relative to one or more needles to be surface treated to produce treated needles 200. In some embodiments, the needle treatment apparatus 400 may be moved/advanced over a surface of a plurality of needles to project ionized airflow 410 over the portion of needle surfaces to be treated or over an entirety of a needle surface to be treated to produce a plurality of treated needles 200. In still another embodiment, a matrix of needles stacked in rows and columns may be treated together by moving needle treatment apparatus 400 relative to a surface of the needles to be treated. Most preferably, bevels 206 of the needles are treated to produce treated needles 200.

In other embodiments, needle treatment apparatus 400 may remain stationary, and a plurality of needles to be treated may be passed through ionized airflow 410 to produce treated needles 200. In such embodiments, a surface treatment using needle treatment apparatus 400 may be a part of an assembly line or manufacturing process used to produce one or more treated needles 200 in-line of the assembly line or manufacturing process for the needles. In these configurations, it will be appreciated that a plurality of treated needles 200 may be produced simultaneously and in bulk. Most preferably, bevels 206 of the needles are treated to produce treated needles 200.

In either a stationary or movable configuration of needle treatment apparatus 400, nozzle portion 404 is preferably disposed in close proximity to surfaces of needles to be treated. Preferably, nozzle portion 404 is disposed around 0.125 to 0.625 inches, more preferably around 0.250 to 0.500 inches, and most preferably around 0.325 to 0.425 inches from a surface of needles to be treated.

Preferably, needle treatment apparatus 400 may be used and/or a surface treatment method according to embodiments herein may be applied to produce one or more treated needles 200 in an open-air environment at atmospheric pressure and does not require the surface treatment to be applied in a closed environment or under vacuum conditions. However, in alternative embodiments, needle treatment apparatus 400 may be used and/or a surface treatment method may be performed in a closed, vacuum-sealed environment. Most preferably, pressurized airflow 408 utilizes ambient air that has been pressurized, but not otherwise treated or purified in any manner. A benefit of some embodiments herein is that no specialized gasses are required for use with needle treatment apparatus 400 and/or a treatment method herein, which can be expensive and dangerous for workers in an area around where treatment is taking place. In other embodiments, a more purified form of gas may be used to create a pressurized airflow 408 and an ionized airflow 410.

In preferred embodiments, ionized airflow 410 may be at a temperature in a range of 225° F. to 375° F., more preferably in a range of 275° F. to 350° F., and most preferably in a range of 300° F. to 325° F. In other embodiments, ionized airflow 410 may be at a temperature of at least about 225° F., at least 250° F., at least 275° F., at least 300° F., at least 325° F., or at least 350° F. In some embodiments, ionized airflow 410 may be at a temperature of not greater than 375° F., not greater than 350° F., not greater than 325° F., not greater than 300° F., not greater than 275° F., not greater than 250° F. It will be appreciated that ionized airflow 410 may be between any of these values, such as at least 225° F. to not greater than 375° F., at least 250° F. to not greater than 350° F., or at least 250° F. to not greater than 300° F. Ionized airflow 410 may be at any temperature within these ranges or within a subrange that overlaps from one recited range to another recited range.

The use of needle treatment apparatus 400 and/or surface treatment methods disclosed herein allow for the bulk production of treated needles 200 in a very fast and efficient method, particularly compared to some prior art treatments that require hours of treatment time. In some embodiments, ionized airflow 410 may be applied to a surface to be treated, most preferably at least bevel 206, for less than 1 second to produce a treated needle 200 and achieve some or all of the benefits disclosed herein. In preferred embodiments, ionized airflow 410 may be applied to a surface to be treated, most preferably at least bevel 206, for a period of time between around 0.05 seconds to 1 second, more preferably around 0.20 seconds and 0.90 seconds, and most preferably around 0.350 seconds and 0.60 seconds. In other embodiments, ionized airflow 410 may be applied to a surface to be treated for at least 0.05 seconds, at least 0.10 seconds, at least 0.15 seconds, at least 0.20 seconds, at least 0.25 seconds, at least 0.30 seconds, at least 0.325 seconds, at least 0.350 seconds, at least 0.375 seconds, at least 0.40 seconds, at least 0.425 seconds, at least 0.450 seconds, at least 0.475 seconds, or at least 0.50 seconds to produce a treated needle 200 and achieve some or all of the benefits disclosed herein. In some embodiments, the surface treatment may be applied for not greater than 1 second, not greater than 0.90 seconds, not greater than 0.80 seconds, not greater than 0.70 seconds, not greater than 0.60 seconds, not greater than 0.50 seconds, not greater than 0.475 seconds, not greater than 0.450 seconds, not greater than 0.425 seconds, not greater than 0.40 seconds, not greater than 0.375 seconds, not greater than 0.350 seconds, or not greater than 0.325 seconds to produce a treated needle 200 and achieve some or all of the benefits disclosed herein. It will be appreciated that the surface treatment may be applied between any of these values, such as at least 0.05 seconds to not greater than 1 second, at least 0.25 seconds to not greater than 0.45 seconds, or even at least 0.30 seconds to not greater than 0.35 seconds to produce a treated needle 200. Ionized airflow 410 may be applied for any period of time within these ranges or within a subrange that overlaps from one recited range to another recited range.

Further, use of needle treatment apparatus 400 and/or surface treatment methods disclosed herein eliminate any need for replacing a drawing needle with a new needle prior to injection. The common practice of utilizing two different needles for a single injection, where a first (or “drawing”) needle is inserted into a vial to draw up the medicine or drug into the syringe, then replacing the drawing needle with a second (or “new”) needle to administer the injection is frequently promoted by syringe manufacturers as a requirement. However, use of needle treatment apparatus 400 and/or surface treatment methods disclosed herein provides enhanced performance in treated needles 200, reducing damage to bevel 206/needle tip 208 from insertion through an elastomeric seal on a medicine vial and removing manufacturing debris from bevel 206. This allows a single treated needle 200 to be used to both draw medicine from a vial and perform an injection and therefore reduces the time, cost, and waste commonly associated with using two needles per injection. The associated cost savings can reduce overall medical costs, making treatment less expensive and available to more patients. Further, supply chain issues that became prevalent during and after the COVID-19 pandemic can be alleviated, since the current demand for replaceable needles used for injections would effectively be reduced by 50%.

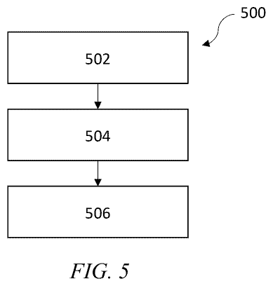

FIG. 5 shows a flowchart of a surface treatment method 500 to enhance the performance of and produce a treated needle 200 according to an embodiment of the disclosure. Surface treatment method 500 may begin at block 502 by providing a needle (or other sharps instrument) to be treated. Surface treatment method 500 may continue at block 504 by subjecting at least a portion of a needle to a surface treatment using an ionized airflow, preferably in ionized airflow 410, in accordance with embodiments disclosed herein. In some embodiments, a surface treatment may be applied to a needle tip, such as needle tip 208, or to an otherwise piercing or cutting edge of a medical sharps instrument. In other embodiments, a surface treatment may be applied to at least a bevel of a needle, such as bevel 206, including a needle tip, such as needle tip 208. In other embodiments, a surface treatment may also be applied to a forward end of a shaft portion of a needle, such as shaft portion 204. In still other embodiments, a surface treatment may be applied to an entirety of a needle. In each of these embodiments a treated needle, such as treated needle 200 is produced. In some embodiments, particularly when performed as part of an in-line manufacturing process for needles, block 504 is preferably carried out prior to applying a lubricant to the needles.

In some embodiments, surface treatment method 500 may further comprise and continue at block 506 by applying a lubricant to treated needles 200. Any standard lubricant used to lubricate medical needles and other medical sharps instruments may be used.

When a needle treatment apparatus 400 and/or surface treatment methods according to embodiments herein are used, performance enhancements may be achieved in treated needles 200 that are produced by those embodiments. In some embodiments, treated needle 200 may comprise a reduced metallic particulate contamination resulting from the needle manufacturing process compared to a conventional needle that has not been subjected to a surface treatment method. In some embodiments, treated needle 200 may experience greater lubrication adherence after undergoing a surface treatment method when compared to a conventional needle that has not been subjected to a surface treatment. In some embodiments, treated needle 200 may comprise a reduced required initial and/or overall penetration force when compared to a conventional needle that has not been subjected to a surface treatment.

### EXAMPLES

Comparative samples C1-C5 of a conventional needle (untreated) and exemplary embodiments S1-S5 of a treated needle 200 were tested. The exemplary treated needle 200 embodiments S1-S5 each received a surface treatment with ionized airflow at a temperature of at least 250° F. to not greater than 350° F. for less than 1 second in accordance with embodiments disclosed herein. The comparative samples C1-C5 were as-manufactured, commercially available needles without any surface treatment. Each needle underwent two sequential insertion processes by inserting each needle through an elastomeric seal (Polyurethane 85 Shore A, 0.4 mm thick, which is a typical seal type used on commercially available vials of medicine) at a penetration rate of 100 millimeters per minute (mm/min) twice. Each needle was fully withdrawn from the elastomeric seal between the first insertion and the second insertion. The first insertion represents the drawing insertion, where a needle may be inserted into a vial to draw up a medicinal fluid into the syringe. The second insertion represents the injection insertion, where the needle may be inserted into a patient or subject to inject the medicinal fluid. Each of C1-C5 and S1-S5 comprised a stainless steel, 25 gauge, ⅝″ long needle coupled to a 3 mL syringe. The needle penetration force was measured and recorded for each needle's first and second insertion. The needle penetration force represents the amount of force required for the needle to initially penetrate the elastomeric seal of the vial.

The first insertion and second insertion measurements of the needle penetration force of comparative samples C1-C5 of the conventional needle without treatment are shown below in Table 1.

TABLE 1
Comparative Samples C1-C5

| | First Insertion | Second Insertion |
|---|---|---|
| | Needle Penetration | Needle Penetration |
| | Force (gf) | Force (gf) |
| C1 | 44.23 | 67.82 |
| C2 | 41.89 | 56.79 |
| C3 | 48.74 | 65.95 |
| C4 | 43.23 | 60.72 |
| C5 | 42.82 | 58.87 |
| Average | 44.18 | 62.03 |

The first and second insertion measurements of the needle penetration force of exemplary embodiments, S1-S5, of treated needle 200 are shown below in Table 2.

TABLE 2
Exemplary Samples S1-S5

| | First Insertion | Second Insertion |
|---|---|---|
| | Needle Penetration | Needle Penetration |
| | Force (gf) | Force (gf) |
| S1 | 42.62 | 60.99 |
| S2 | 42.59 | 57.75 |
| S3 | 43.26 | 57.59 |
| S4 | 49.37 | 57.49 |
| S5 | 45.29 | 57.98 |
| Average | 44.63 | 58.36 |

### First Insertion.

As shown, the average first insertion needle penetration force of the comparative samples C1-C5 was slightly better than that of the exemplary embodiments S1-S5. It will be appreciated that the average values of 44.18 gf and 44.63 gf, respectively, are relatively similar (about 1.02% difference) and these results are generally considered comparable. The slight differences may be attributable to tolerances in the manufacturing process or other variances. Further, the shaft portions of C1-C5 and S1-S5 remain untreated, so some residual lubricant or other anomalies may slightly affect the results. Additionally, the force results for the second insertion, particularly the penetration force of the second insertion, are more indicative of benefits achieved by us of a needle treatment apparatus 400 and/or surface treatment methods according to embodiments herein, as they indicate that it is not necessary to replace the draw needle with a new needle prior to injection.

### Second Insertion.

As shown, the average second insertion needle penetration force of the comparative samples C1-C5 was 62.03 gf, while the average second insertion needle penetration force of the exemplary embodiments S1-S5 was 58.36 gf. It will be appreciated that the exemplary embodiments S1-S5 show a 5.92% reduction in the second insertion needle penetration force after receiving the surface treatment, which is generally considered significant. Without being bound by theory, the reduced needle penetration force of the exemplary embodiments S1-S5 may be attributable to the surface treatment removing at least some of the residual metallic particulate resulting from the needle manufacturing process (the process upstream of surface treatment according to embodiments herein) and/or an increased adherence of the lubrication applied to the treated needles 200 that provide for a smoother second insertion while the needles 200 penetrate the elastomeric seal on the vial. Additionally, in some embodiments, the reduced needle penetration force of the exemplary embodiments S1-S5 may be attributable to the surface treatment strengthening or tempering the treated needle 200, such that the treated needle 200 maintains needle tip integrity as compared to the comparative samples C1-C5 of the conventional needle without treatment.

It will be appreciated that the second insertion needle penetration force is the most important aspect for a needle, since it represents the force required to pierce or puncture the skin of a patient when administering an injection. Since the second insertion needle penetration force is the “stick” a patient experiences when receiving an injection, a reduced second insertion needle penetration force provided by treated needle 200 will result in easier needle penetration into the skin with less resistance, which will drastically decrease the pain experienced by many patients when receiving an injection. Further, while only a single gauge treated needle 200 was tested, in some embodiments, the reduction in second insertion needle penetration force may be greater for other needle sizes. Accordingly, in some embodiments, treated needle 200 may reduce the needle penetration force for a second insertion by at least 1%, at least 2%, at least 3%, at least 4%, at least 5%, at least 6%, at least 7%, at least 8%, at least 9%, at least 10%, at least 15%, or even greater over a penetration force for a second insertion of a conventional needle without treatment.

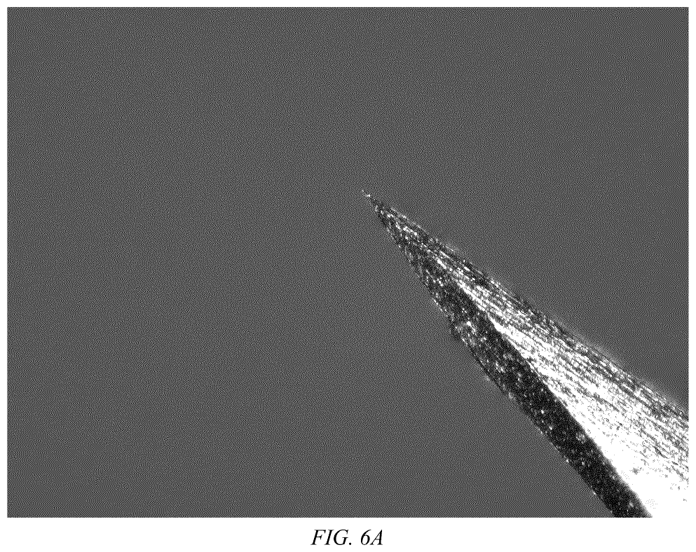

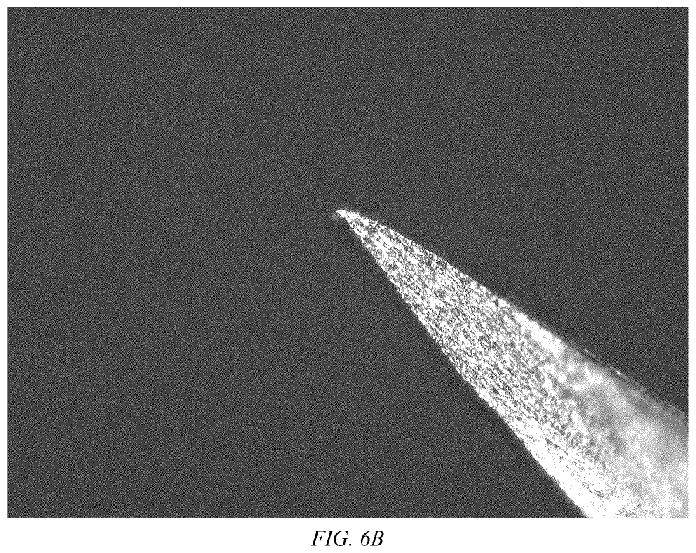

FIG. 6A shows an image of a needle tip of a comparative sample of a conventional needle without treatment prior to undergoing a first insertion process, and FIG. 6B shows an image of the needle tip of the same comparative sample of the conventional needle without treatment after undergoing the first insertion process. As shown in FIG. 6A, the comparative sample the conventional needle without treatment comprises a sharp needle tip before the first insertion. This may contribute to the comparative samples C1-C5 having similar first insertion needle penetration as compared to exemplary embodiments S1-S5 of treated needle 200. However, as shown in FIG. 6B, the needle tip is slightly bent and dulled after the first insertion. This may contribute to higher second insertion needle penetration force in comparative samples C1-C5 as compared to exemplary embodiments, S1-S5, of treated needle 200.

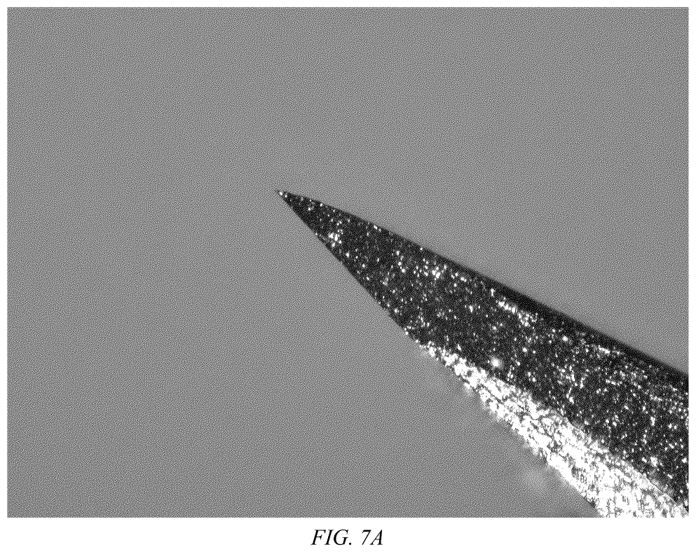

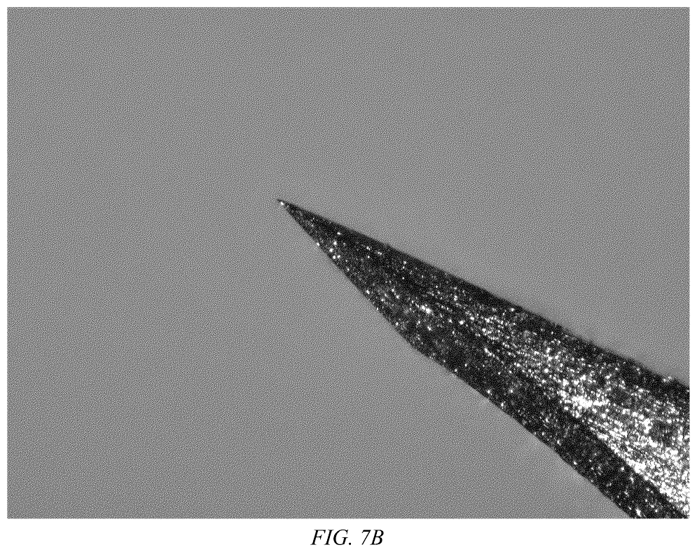

FIG. 7A shows an image of a needle tip of an exemplary embodiment of a treated needle 200 according to an embodiment of the disclosure prior to undergoing a first insertion process, and FIG. 7B shows an image of a needle tip of the same exemplary embodiment of the treated needle 200 according to an embodiment of the disclosure after undergoing the first insertion process. As shown in FIGS. 7A-7B, the exemplary embodiment of a treated needle 200 comprises a sharp needle tip before and after the first insertion. The needle tip of treated needle 200 is still extremely sharp and does not experience the bending or dulling that the comparative sample shown in FIG. 6B caused by the first insertion. Further, the reduced damage to the needle tip for treated needle 200 may contribute to the lower second insertion needle penetration force in the exemplary embodiments S1-S5 of treated needle 200 as compared to the comparative samples C1-C5.

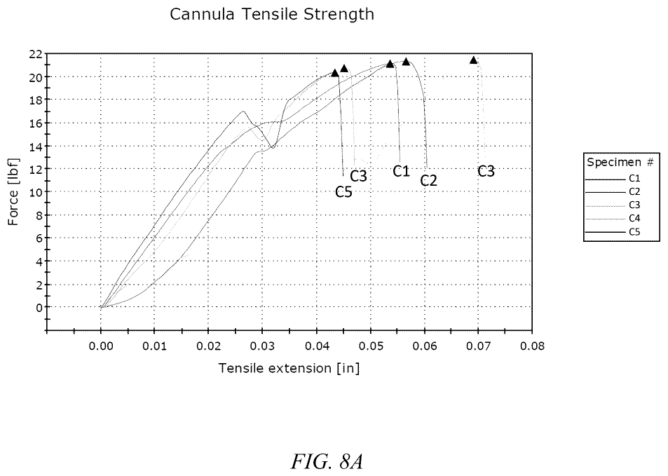

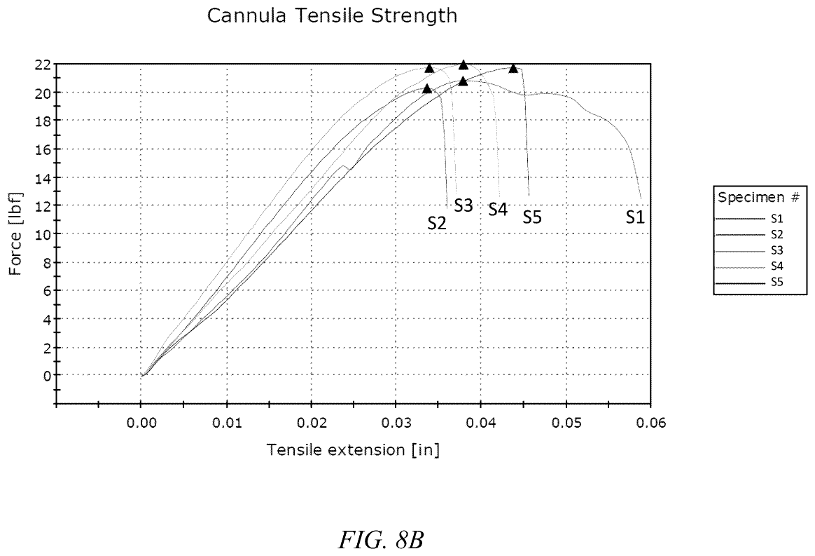

FIG. 8A shows a graph of peak force versus tensile extension of a plurality of comparative samples of conventional needles without treatment, and FIG. 8B shows a graph of peak force versus tensile extension of a plurality of exemplary embodiments of treated needles 200 according to an embodiment of the disclosure.

The peak force and tensile extension of comparative samples C1-C5 of the conventional needle without treatment are shown in Table 3 below.

TABLE 3
Untreated Samples C1-C5

| | | Tensile Extension at |
|---|---|---|
| | Peak Force (lbf) | Peak Force (in) |
| C1 | 21.1 | 0.05356 |
| C2 | 21.3 | 0.05655 |
| C3 | 21.5 | 0.06903 |
| C4 | 20.7 | 0.04507 |
| C5 | 20.4 | 0.04337 |
| Average | 21.0 | 0.05352 |

The peak force and tensile extension of exemplary embodiments S1-S5 of the treated needle 200 are shown in Table 4 below.

TABLE 4
Treated Samples S1-S5

| | | Tensile Extension at |
|---|---|---|
| | Peak Force (lbf) | Peak Force (in) |
| S1 | 20.8 | 0.03783 |
| S2 | 20.3 | 0.03359 |
| S3 | 21.7 | 0.03386 |
| S4 | 22.0 | 0.03786 |
| S5 | 21.7 | 0.04373 |
| Average | 21.3 | 0.03737 |

The average tensile extension of the comparative samples C1-C5 was 0.05352 inches, while the average tensile extension of the exemplary embodiments S1-S5 was 0.03737 inches. It will be appreciated that the exemplary embodiments S1-S5 show a 30.18% reduction in the tensile extension force after receiving a surface treatment according to a preferred embodiment herein. This demonstrates the exemplary embodiments S1-S5 of the treated needle 200 maintain their structural integrity much better than the comparative samples C1-C5 of the conventional needle without treatment, which may contribute to the reduction in second insertion penetration force and absence of deformation of the cannula tips of the exemplary embodiments S1-S5 of the treated needle 200 as the resistance of strain about the cannula axis has increased.

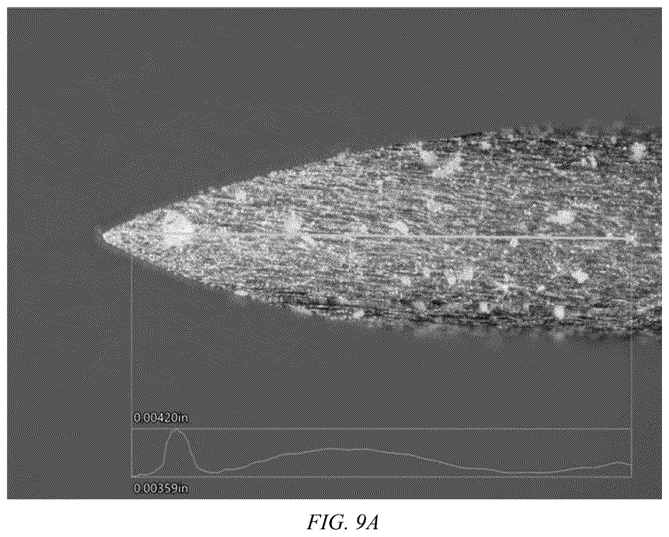

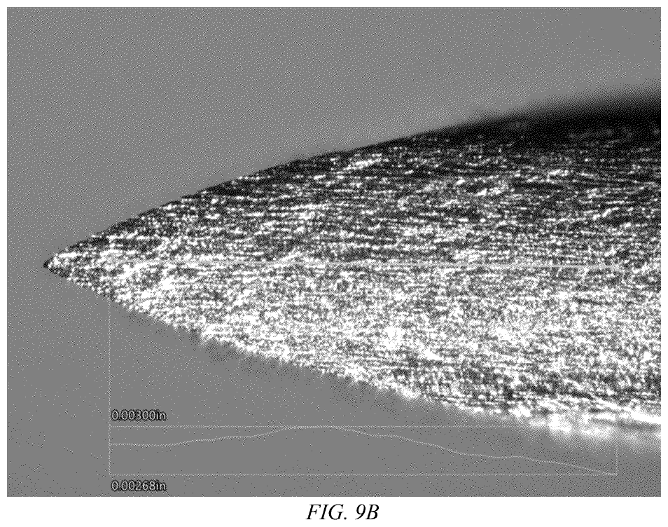

FIG. 9A shows an image of a needle tip that has not had a surface treatment according to an embodiment of the disclosure applied. FIG. 9B shows an image of the same needle tip from FIG. 9A after undergoing a surface treatment according to a preferred embodiment of the disclosure. These images are overlaid with a graph showing the height or profile of contaminants (such as metallic particulate matter) present on the untreated needle tip (FIG. 9A) and treated needle tip (FIG. 9B), allowing for increased surface exposure for the adhesion of lubricant. As shown, the contamination present on the untreated needle (FIG. 9A) includes metallic particulate ranging in height between 0.00359 inches to 0.0042 inches, while the contamination present on the treated needle 200 includes metallic particulate ranging between 0.00268 to 0.00300 inches. This demonstrates a reduction in contaminants present on the treated needle 200. Such a reduction of contaminants would further contribute to the reduction in second insertion penetration force demonstrated by the exemplary embodiments S1-S5 of the treated needle 200.

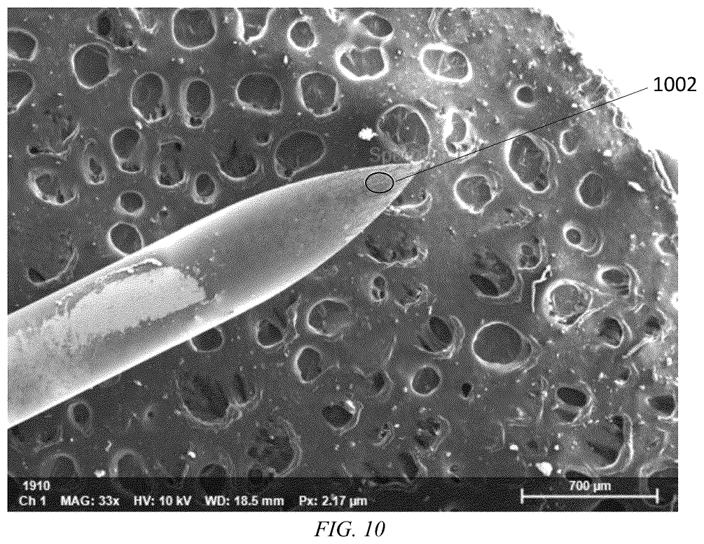

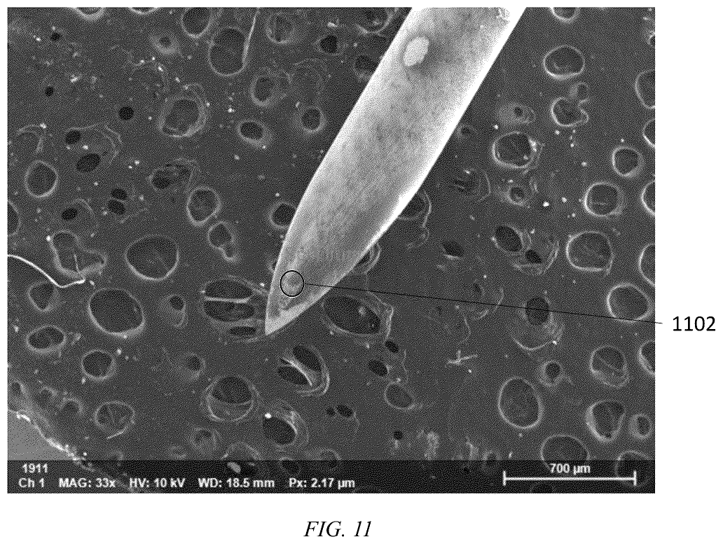

Comparative sample C6 of a conventional untreated needle and an exemplary embodiment S6 of a treated needle 200 received a scanning electron microscopy/energy-dispersive X-ray spectrometry (SEM-EDS) analysis to determine the elemental composition of the surface of each needle. Each of C6 and S6 comprised a stainless steel, 25 gauge, ⅝″ long needle coupled to a 3 mL syringe. The comparative sample C6 remained untreated. The exemplary embodiment S6 received a surface treatment with ionized airflow at a temperature of at least 250° F. to not greater than 350° F. for less than 1 second in accordance with embodiments disclosed herein. The needle tips were cut from their respective syringes and adhered to an aluminum SEM sample holder using carbon tape. The analysis was performed on the needle tip of each of C6 and S6 opposite the bevel. FIG. 10 shows an energy-dispersive X-ray spectrometry (EDS) image of the needle tip of the comparative sample C6, showing the sampled area 1002 subjected to the SEM-EDS analysis. FIG. 11 shows an energy-dispersive X-ray spectrometry (EDS) image of the needle tip of the exemplary embodiment S6, showing the sampled area 1102 subjected to the SEM-EDS analysis.

The EDS spectrum analysis revealed that the needle tip of the comparative sample C6 comprised silicon, chromium and iron. The normalized mass percentages of each of the major elements found on the surface of the needle tip of the comparative sample C6 are shown in Table below.

TABLE 5
Untreated Comparative Sample C6

| Element | Spectrum 1 Normalized Mass Percentage |
|---|---|
| Silicon | 43.79 |
| Chromium | 6.09 |
| Iron | 50.12 |

The EDS spectrum analysis revealed that the needle tip of the treated exemplary embodiment S6 comprised silicon, chromium, manganese, and iron. The normalized mass percentages of each of the major elements found on the surface of the needle tip of the exemplary embodiment S6 are shown in Table below.

TABLE 6
Treated Exemplary Embodiment S6

| Element | Spectrum 1 Normalized Mass Percentage |
|---|---|
| Silicon | 45.82 |
| Chromium | 5.36 |
| Manganese | 1.54 |
| Iron | 47.29 |

Most notably, the comparative sample C6 contained a normalized mass percentage of silicon of 43.79%, and the exemplary embodiment S6 contained a normalized mass percentage of silicon of 45.82%. This represents an increase of 2.03% of silicon in the exemplary embodiment S6. The increased amount of silicon, which is used to make silicone, represents an increase in adhesion of a lubricant (e.g., silicone) to the exemplary embodiment S6 of the treated needle 200 as compared to the comparative sample C6 of the conventional untreated needle. This provides the exemplary embodiment S6 of the treated needle 200 with a smoother initial and continued penetration into an elastomeric seal of a medicine vial, a patient's skin, or both. The increased adhesion of the silicone lubricant also provides the exemplary embodiment S6 of the treated needle 200 with a reduced “stick” feeling upon initial insertion of the needle tip into the patient and a lower drag force as the treated needle 200 is continually inserted deeper into the patient's skin as compared to the comparative sample C6 of the conventional untreated needle, thereby reducing and/or altogether eliminating any pain felt by the patient. Thus, the treated needle 200 provides exceptional benefits over a conventional untreated needle.

Although primarily described herein with respect to a needle for a syringe, and particularly a syringe that utilizes a replaceable needle, needle treatment apparatus 400 and/or surface treatment methods according to embodiments herein may be used with any type of medical needle, including on blood tube collection holders, blood tube sets, IV catheters, injection pens, syringes with retractable needles, syringes with needles that are not removable (or not designed to be removed), and suture needles, as well as other medical sharps instruments, such as scalpels and razor blades.

It will be appreciated that a syringe 100, a treated needle 200, and/or a method 500 disclosed herein may include one or more of the following embodiments:

Embodiment 1. A method of enhancing performance of a needle, comprising: providing a needle; and administering a treatment to at least a portion of the needle by projecting a heated, ionized airflow over the at least a portion of the needle at a temperature of at least 225° F. to not greater than 325° F. for not greater than 1 second, wherein the treatment reduces a post-draw needle penetration force of the needle as compared to a traditional untreated needle.

Embodiment 2. The method of embodiment 1, further comprising: administering the treatment in an open-air environment at atmospheric pressure.

Embodiment 3. The method of any of embodiments 1 to 2, further comprising: administering the treatment for at least 0.05 seconds, at least 0.10 seconds, at least 0.15 seconds, at least 0.20 seconds, at least 0.25 seconds, at least 0.30 seconds, at least 0.325 seconds, at least 0.350 seconds, at least 0.375 seconds, at least 0.40 seconds, at least 0.425 seconds, at least 0.450 seconds, at least 0.475 seconds, or at least 0.50 seconds per needle.

Embodiment 4. The method of any of embodiments 1 to 3, further comprising: administering the treatment for not greater than 1 second, not greater than 0.90 seconds, not greater than 0.80 seconds, not greater than 0.70 seconds, not greater than 0.60 seconds, not greater than 0.50 seconds, not greater than 0.475 seconds, not greater than 0.450 seconds, not greater than 0.425 seconds, not greater than 0.40 seconds, not greater than 0.375 seconds, not greater than 0.350 seconds, or not greater than 0.325 seconds per needle.

Embodiment 5. The method of embodiment 1 to 4, further comprising: administering the treatment with a needle treatment apparatus comprising a housing coupled to an air supply source and comprising a nozzle portion and an electrode that extends in proximity to the nozzle portion, wherein the electrode is configured to ionize an airflow passing through the housing to produce the heated, ionized airflow.

Embodiment 6. The method of embodiment 5, wherein the needle treatment apparatus is movable and configured to administer the treatment to a plurality of needles simultaneously.

Embodiment 7. The method of any of embodiments 5 to 6, wherein the needle treatment apparatus remains stationary as part of an assembly line or manufacturing process to administer the treatment to a plurality of needles that are passed through the heated, ionized airflow.

Embodiment 8. The method of any of embodiments 1 to 7, further comprising: reducing an amount of residual metallic particulate from the needle that adheres to an outer surface of the needle during manufacturing of the needle as compared to a traditional untreated needle.

Embodiment 9. The method of embodiment 8, wherein the residual metallic particulate on the outer surface of the needle comprises a height of not greater than 0.00300 inches.

Embodiment 10. The method of any of embodiments 8 to 9, wherein the residual metallic particulate on the outer surface of the needle is reduced by at least 1%, at least 2%, at least 3%, at least 4%, at least 5%, at least 10%, at least 15%, at least 20%, or even at least 25%.

Embodiment 11. The method of any of embodiments 8 to 10, wherein reducing the amount of residual metallic particulate from the needle is accomplished by the heated, ionized airflow blowing residual metallic particulate from an outer surface of the needle, the heated, ionized airflow causing the residual metallic particulate to release from the outer surface of the needle, or a combination thereof.

Embodiment 12. The method of any of embodiments 1 to 11, further comprising: applying a lubricant to the needle after administering the treatment to the needle.

Embodiment 13. The method of embodiment 12, wherein the treatment increases adherence of the lubricant to the needle as compared to a traditional untreated needle.

Embodiment 14. The method of any of embodiments 12 to 13, wherein the treatment increases the adherence of the lubricant to the needle by at least 1%, at least 2%, at least 3%, at least 4%, at least 5%, at least 10%, at least 15%, at least 20%, or even at least 25%.

Embodiment 15. The method of any of embodiments 1 to 14, wherein the treatment provides a needle tip of the needle with a greater strength.

Embodiment 16. The method of embodiment 15, further comprising: reducing a tensile extension of the needle by at least 5%, at least 10%, at least 15%, at least 20%, at least 25%, or at least 30% as compared to a traditional untreated needle.

Embodiment 17. The method of any of embodiments 1 to 16, wherein the treatment reduces the post-draw needle penetration force by at least 1%, at least 2%, at least 3%, at least 4%, at least 5%, at least 6%, at least 7%, at least 8%, at least 9%, or even at least 10%.

Embodiment 18. The method of any of embodiments 1 to 17, wherein the post-draw needle penetration force is not greater than 61.0 gf for a 25-gauge needle.

Embodiment 19. The method of any of embodiments 1 to 18, wherein the needle is formed from stainless steel or titanium.

Embodiment 20. The method of any of embodiments 1 to 19, wherein the needle is a component of a needle assembly that is coupled to a syringe.

Embodiment 21. A needle, comprising: a base; a shaft portion; and a bevel forming a needle tip; wherein a treatment is administered to at least a portion of the needle by projecting a heated, ionized airflow over the at least a portion of the needle at a temperature of at least 225° F. to not greater than 325° F. for not greater than 1 second, wherein the treatment reduces a post-draw needle penetration force of the needle as compared to a traditional untreated needle.

Embodiment 22. The needle of claim 21, wherein the treatment is administered in an open-air environment at atmospheric pressure.

Embodiment 23. The needle of claim 21, wherein the treatment is administered for at least 0.05 seconds, at least 0.10 seconds, at least 0.15 seconds, at least 0.20 seconds, at least 0.25 seconds, at least 0.30 seconds, at least 0.325 seconds, at least 0.350 seconds, at least 0.375 seconds, at least 0.40 seconds, at least 0.425 seconds, at least 0.450 seconds, at least 0.475 seconds, or at least 0.50 seconds per needle.

Embodiment 24. The needle of claim 23, wherein the treatment is administered not greater than 1 second, not greater than 0.90 seconds, not greater than 0.80 seconds, not greater than 0.70 seconds, not greater than 0.60 seconds, not greater than 0.50 seconds, not greater than 0.475 seconds, not greater than 0.450 seconds, not greater than 0.425 seconds, not greater than 0.40 seconds, not greater than 0.375 seconds, not greater than 0.350 seconds, or not greater than 0.325 seconds per needle.

Embodiment 25. The needle of claim 21, wherein the treatment is administered with a needle treatment apparatus comprising a housing coupled to an air supply source and comprising a nozzle portion and an electrode that extends in proximity to the nozzle portion, wherein the electrode is configured to ionize an airflow passing through the housing to produce the heated, ionized airflow.

Embodiment 26. The needle of claim 25, wherein the needle treatment apparatus is movable and configured to administer the treatment to a plurality of needles simultaneously.

Embodiment 27. The needle of claim 25, wherein the needle treatment apparatus remains stationary as part of an assembly line or manufacturing process to administer the treatment to a plurality of needles that are passed through the heated, ionized airflow.

Embodiment 28. The needle of claim 21, wherein the treatment reduces an amount of residual metallic particulate from the needle that adheres to an outer surface of the needle during manufacturing of the needle as compared to a traditional untreated needle.

Embodiment 29. The needle of claim 28, wherein the residual metallic particulate on the outer surface of the needle comprises a height of not greater than 0.00300 inches.

Embodiment 30. The needle of claim 29, wherein the residual metallic particulate on the outer surface of the needle is reduced by at least 1%, at least 2%, at least 3%, at least 4%, at least 5%, at least 10%, at least 15%, at least 20%, or even at least 25%.

Embodiment 31. The needle of claim 30, wherein the residual metallic particulate is reduced by the heated, ionized airflow blowing residual metallic particulate from an outer surface of the needle, the heated, ionized airflow causing the residual metallic particulate to release from the outer surface of the needle, or a combination thereof.

Embodiment 32. The needle of claim 21, wherein a lubricant is applied to the needle after the treatment.

Embodiment 33. The needle of claim 32, wherein the treatment increases adherence of the lubricant to the needle as compared to a traditional untreated needle.

Embodiment 34. The needle of claim 33, wherein the treatment increases the adherence of the lubricant to the needle by at least 1%, at least 2%, at least 3%, at least 4%, at least 5%, at least 10%, at least 15%, at least 20%, or even at least 25%.

Embodiment 35. The needle of claim 21, wherein the treatment provides the needle tip of the needle with a greater strength.

Embodiment 36. The needle of claim 35, wherein the treatment reduces a tensile extension of the needle by at least 5%, at least 10%, at least 15%, at least 20%, at least 25%, or at least 30% as compared to a traditional untreated needle.

Embodiment 37. The needle of claim 21, wherein the treatment reduces the post-draw needle penetration force by at least 1%, at least 2%, at least 3%, at least 4%, at least 5%, at least 6%, at least 7%, at least 8%, at least 9%, or even at least 10%.

Embodiment 38. The needle of claim 37, wherein the post-draw needle penetration force is not greater than 61.0 gf for a 25-gauge needle.

Embodiment 39. The needle of claim 21, wherein the needle is formed from stainless steel or titanium.

Embodiment 40. The needle of claim 39, wherein the needle is a component of a needle assembly that is coupled to a syringe.

Embodiment 41. A syringe, comprising: a barrel comprising a cylindrical tubular body and a nose end; a plunger disposed within the cylindrical tubular body of the barrel; and a needle assembly coupled to the barrel and extending from the nose end of the barrel, the needle assembly comprising a needle holder and a needle comprising a base, a shaft portion, and a bevel forming a needle tip, wherein a treatment is administered to at least a portion of the needle by projecting a heated, ionized airflow over the at least a portion of the needle at a temperature of at least 225° F. to not greater than 325° F. for not greater than 1 second, wherein the treatment reduces a post-draw needle penetration force of the needle as compared to a traditional untreated needle.

Embodiment 42. The syringe of claim 41, wherein the treatment is administered in an open-air environment at atmospheric pressure.

Embodiment 43. The syringe of claim 41, wherein the treatment is administered for at least 0.05 seconds, at least 0.10 seconds, at least 0.15 seconds, at least 0.20 seconds, at least 0.25 seconds, at least 0.30 seconds, at least 0.325 seconds, at least 0.350 seconds, at least 0.375 seconds, at least 0.40 seconds, at least 0.425 seconds, at least 0.450 seconds, at least 0.475 seconds, or at least 0.50 seconds per needle.

Embodiment 44. The syringe of claim 43, wherein the treatment is administered not greater than 1 second, not greater than 0.90 seconds, not greater than 0.80 seconds, not greater than 0.70 seconds, not greater than 0.60 seconds, not greater than 0.50 seconds, not greater than 0.475 seconds, not greater than 0.450 seconds, not greater than 0.425 seconds, not greater than 0.40 seconds, not greater than 0.375 seconds, not greater than 0.350 seconds, or not greater than 0.325 seconds per needle.

Embodiment 45. The syringe of claim 41, wherein the treatment is administered with a needle treatment apparatus comprising a housing coupled to an air supply source and comprising a nozzle portion and an electrode that extends in proximity to the nozzle portion, wherein the electrode is configured to ionize an airflow passing through the housing to produce the heated, ionized airflow.

Embodiment 46. The syringe of claim 45, wherein the needle treatment apparatus is movable and configured to administer the treatment to a plurality of needles simultaneously.

Embodiment 47. The syringe of claim 45, wherein the needle treatment apparatus remains stationary as part of an assembly line or manufacturing process to administer the treatment to a plurality of needles that are passed through the heated, ionized airflow.

Embodiment 48. The syringe of claim 41, wherein the treatment reduces an amount of residual metallic particulate from the needle that adheres to an outer surface of the needle during manufacturing of the needle as compared to a traditional untreated needle.

Embodiment 49. The syringe of claim 48, wherein the residual metallic particulate on the outer surface of the needle comprises a height of not greater than 0.00300 inches.

Embodiment 50. The syringe of claim 49, wherein the residual metallic particulate on the outer surface of the needle is reduced by at least 1%, at least 2%, at least 3%, at least 4%, at least 5%, at least 10%, at least 15%, at least 20%, or even at least 25%.

Embodiment 51. The syringe of claim 50, wherein the residual metallic particulate is reduced by the heated, ionized airflow blowing residual metallic particulate from an outer surface of the needle, the heated, ionized airflow causing the residual metallic particulate to release from the outer surface of the needle, or a combination thereof.

Embodiment 52. The syringe of claim 41, wherein a lubricant is applied to the needle after the treatment.

Embodiment 53. The syringe of claim 52, wherein the treatment increases adherence of the lubricant to the needle as compared to a traditional untreated needle.

Embodiment 54. The syringe of claim 53, wherein the treatment increases the adherence of the lubricant to the needle by at least 1%, at least 2%, at least 3%, at least 4%, at least 5%, at least 10%, at least 15%, at least 20%, or even at least 25%.

Embodiment 55. The syringe of claim 41, wherein the treatment provides the needle tip of the needle with a greater strength.

Embodiment 56. The syringe of claim 55, wherein the treatment reduces a tensile extension of the needle by at least 5%, at least 10%, at least 15%, at least 20%, at least 25%, or at least 30% as compared to a traditional untreated needle.

Embodiment 57. The syringe of claim 41, wherein the treatment reduces the post-draw needle penetration force by at least 1%, at least 2%, at least 3%, at least 4%, at least 5%, at least 6%, at least 7%, at least 8%, at least 9%, or even at least 10%.

Embodiment 58. The syringe of claim 57, wherein the post-draw needle penetration force is not greater than 61.0 gf for a 25-gauge needle.

Embodiment 59. The syringe of claim 41, wherein the needle is formed from stainless steel or titanium.

In the foregoing specification, the concepts have been described with reference to specific embodiments. However, those of ordinary skill in the art appreciate that various modifications and changes can be made without departing from the scope of the invention as set forth in the claims below. Other examples that occur to those skilled in the art are intended to be within the scope of the claims if they have structural elements that do not differ from the literal language of the claims, or if they include equivalent structural elements with insubstantial differences from the literal languages of the claims. Accordingly, the specification and figures are to be regarded in an illustrative rather than in a restrictive sense, and all such modifications are intended to be included within the scope of the invention.

References to “about” or “around” with respect to dimensions generally mean +/−0.10 inches for dimensions indicated to two decimal places; +/−0.005 for dimensions indicated to three or more decimal places; and +/−1 for percentages. Further, references to numerical values stated in ranges include each and every value within that range and any and all subset combinations within ranges, including subsets that overlap from one preferred range to a more preferred range and even if the specific subset of the range is not specifically described herein.

One of ordinary skill in the art will understand that not all of the steps of methods or features of systems and apparatus described above in the detailed description or in the examples are required, that a portion of a specific step or feature may not be required, and that one or more further steps or features may be performed or included in addition to those described.

As used herein, the terms “comprises,” “comprising,” “includes,” “including,” “has,” “having,” or any other variation thereof, are intended to cover a non-exclusive inclusion. For example, a process, method, article, or apparatus that comprises a list of features is not necessarily limited only to those features but may include other features not expressly listed or inherent to such process, method, article, or apparatus. Further, unless expressly stated to the contrary, “of” refers to an inclusive-or and not to an exclusive-or. For example, a condition A or B is satisfied by any one of the following: (1) A is true (or present), and B is false (or not present), (2) A is false (or not present), and B is true (or present), and (3) both A and B are true (or present).

Also, the use of “a” or “an” is employed to describe elements and components described herein. This is done merely for convenience and to give a general sense of the scope of the invention. This description should be read to include one or at least one, and the singular also includes the plural unless it is obvious that it is meant otherwise.

Benefits, other advantages, and solutions to problems have been described above with respect to one or more specific embodiments. After reading the specification, those of ordinary skill in the art will appreciate that certain features that are, for clarity, described herein in the context of separate embodiments, may also be provided in combination in a single embodiment. Conversely, various features that are, for brevity, described in the context of a single embodiment, may also be provided separately or in any subcombination. Any feature, component, element, or method steps of an embodiment herein may be used with any other features, components, elements, or steps of other embodiments even if not specifically described with respect to that embodiment, unless such combination is explicitly excluded herein. Any feature, component, element, or method steps described as excluded with any particular preferred embodiment herein may similarly be excluded with any other preferred embodiment herein even if not specifically described with such embodiment.

What is claimed is:

1. A treated medical needle, comprising:
a base;
a shaft; and
a treated bevel forming a needle tip, wherein the treated bevel comprises a conventional bevel that has received a treatment, wherein the treatment comprises administering an airflow having a temperature in a range of 225° F. to 325° F. for a treatment period of at least 0.05 seconds to not greater than 1.0 second to the conventional bevel to produce the treated bevel;
wherein the treated medical needle has a second insertion needle penetration force that is less than a second insertion needle penetration force of a similar conventional needle that has not received the treatment.

2. The treated medical needle of claim 1, wherein the treatment is administered in an open-air environment at atmospheric pressure.

3. The treated medical needle of claim 2, wherein the airflow is ionized and comprises a pressure of about 60 psi to about 110 psi.

4. The treated medical needle of claim 1, wherein the treated bevel has a reduced amount of residual metallic particulate as compared to the conventional bevel that has not received the treatment.

5. The treated medical needle of claim 4, wherein the residual metallic particulate on treated bevel comprises a height of not greater than 0.00300 inches.

6. The treated medical needle of claim 1, further comprising: an amount of lubricant applied on an exterior surface of the treated bevel and an exterior surface of at least a portion of the shaft.

7. The treated medical needle of claim 6, wherein the treated medical needle comprises an increased adherence of the lubricant as compared to the conventional needle that has not received the treatment by at least 1%, at least 2%, at least 3%, at least 4%, at least 5%, at least 10%, at least 15%, at least 20%, or even at least 25%.

8. The treated medical needle of claim 1, wherein the treatment provides the treated bevel with a greater strength as compared to the conventional needle that has not received the treatment.

9. The treated medical needle of claim 8, wherein the needle tip of the treated bevel is not bent after a second insertion of the treated medical needle through an elastomeric seal on a medicine vial, through a patient's skin, or a combination thereof.

10. The treated medical needle of claim 9, wherein treating the medical needle reduces the second insertion needle penetration force by at least 1%, at least 2%, at least 3%, at least 4%, at least 5%, at least 6%, at least 7%, at least 8%, at least 9%, or even at least 10%.

11. The treated medical needle of claim 10, wherein the treated medical needle has a gauge of 25, and wherein the second insertion needle penetration force of the treated medical needle is not greater than 61.0 gf.

12. The treated medical needle of claim 1, wherein the medical needle is formed from stainless steel or titanium.

13. The treated medical needle of claim 12, wherein the treated medical needle is a component of a needle assembly that is coupled to a syringe.

14. The treated medical needle of claim 1, wherein the treatment is applied by a treatment system comprising a compressor to compress the ambient air, a housing coupled to the compressor, and a heating element disposed within the housing and configured to heat the ambient air.

15. The treated medical needle of claim 14, wherein the heating element comprises an electrode configured to ionize the airflow as it passes passing through the housing to produce an ionized airflow.

16. A method of enhancing performance of a medical needle, comprising:
providing a medical needle; and
treating the medical needle by administering an airflow to at least a portion of the medical needle to produce a treated medical needle, wherein the airflow comprises ambient air heated to a temperature in a range of 225° F. to 325° F., and wherein the medical needle is exposed to the airflow for a treatment period of at least 0.05 seconds to not greater than 1.0 second.

17. The method of claim 16, wherein treating the medical needle occurs in an open-air environment at atmospheric pressure.

18. The method of claim 16, further comprising: generating the airflow using a treatment system comprising a compressor to compress the ambient air, a housing coupled to the compressor, and a heating element disposed within the housing and configured to heat the ambient air.

19. The method of claim 18, wherein the heating element comprises an electrode configured to ionize the airflow as it passes passing through the housing to produce an ionized airflow.

20. The method of claim 19, wherein the airflow comprises a pressure of about 60 psi to about 110 psi.

21. The method of claim 20, wherein the treatment system is movable and configured to project the airflow to a plurality of medical needles simultaneously.

22. The method of claim 20, wherein the treatment system remains stationary as part of an assembly line or manufacturing process for a plurality of medical needles and is configured to project the airflow as the plurality of the medical needles pass by the treatment system.

23. The method of claim 20, wherein the medical needle has a first amount of a residual metallic particulate adhered to an outer surface of the medical needle from manufacturing, wherein the treated medical needle has a second amount of the residual metallic particulate, and wherein the first amount is greater than the second amount by at least 1%, at least 2%, at least 3%, at least 4%, at least 5%, at least 10%, at least 15%, at least 20%, or even at least 25%.

24. The method of claim 16, further comprising: reducing an amount of residual metallic particulate from the treated medical needle that adheres to an outer surface of the medical needle during manufacturing of the medical needle as compared to a traditional untreated needle.

25. The method of claim 24, wherein the residual metallic particulate on the outer surface of the treated medical needle comprises a height of not greater than 0.00300 inches.

26. The method of claim 16, further comprising: applying a lubricant to the treated medical needle after treating the medical needle, wherein treating the medical needle increases adherence of the lubricant to the treated medical needle as compared to a traditional untreated needle by at least 1%, at least 2%, at least 3%, at least 4%, at least 5%, at least 10%, at least 15%, at least 20%, or even at least 25%.

27. The method of claim 16, wherein treating the medical needle provides a needle tip of the medical needle with a greater strength as compared to a traditional untreated needle.

28. The method of claim 16, wherein treating the medical needle reduces a post-draw needle penetration force by at least 1%, at least 2%, at least 3%, at least 4%, at least 5%, at least 6%, at least 7%, at least 8%, at least 9%, or even at least 10%.

29. The method of claim 28, wherein the post-draw needle penetration force is not greater than 61.0 gf for a 25-gauge needle.

30. The method of claim 16, wherein the medical needle is formed from stainless steel or titanium.
